(19) United States
(12) Patent Application Publication
(10) Pub. No.: US 2003/0149058 A1
(43) Pub. Date: Aug. 7, 2003

(54) Use of dipyridamole or mopidamol for treatment and prevention of fibrin-dependent microcirculation disorders

(76) Inventor: Wolfgang Eisert, Hannover (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/376,072

(22) Filed: Feb. 27, 2003

Related U.S. Application Data
(63) Continuation of application No. 09/694,610, filed on Oct. 23, 2000, now abandoned.
(60) Provisional application No. 60/167,797, filed on Nov. 29, 1999.

(30) Foreign Application Priority Data
Oct. 22, 1999 (DE) 991211210

(51) Int. Cl. A61K 31/519; A61K 31/423; A61K 31/366; A61K 31/225

(52) U.S. Cl. 514/262.1; 514/423; 514/460; 514/548

(57) ABSTRACT

A method of treatment of the human or non-human animal body for treating fibrin-dependent microcirculation disorders is disclosed, for example, microcirculation disorders caused by metabolic diseases, inflammatory reactions or autoimmune diseases; peripheral microcirculation disorders or microcirculation disorders associated with increased cell fragmentation comprising administering to a human or non-human animal body in need of such treatment an effective amount of a pharmaceutical composition containing a pyrimido-pyrimidine selected from dipyridamole, mopidamol and the pharmaceutically acceptable salts thereof, and the use of said pyrimido-pyrimidine for the manufacture of a corresponding pharmaceutical composition.

### RELATED APPLICATIONS

This application is a continuation of U.S. Ser. No. 09/694,610, filed Oct. 23, 2000, which claims, as does the present application, priority to U.S. provisional application Serial No. 60/167,797, filed November 1999, the disclosures of all of which are incorporated by reference in their entireties.

### FIELD OF THE INVENTION

This invention relates to a method of treating fibrin-dependent microcirculation disorders using dipyridamole or mopidamol as active principle, providing a lasting improvement of microcirculation under treatment, and the use of dipyridamole or mopidamol for the manufacture of a corresponding pharmaceutical composition.

### BACKGROUND OF THE INVENTION

Dipyridamole {2,6-bis(diethanolamino)-4,8-dipiperidino-pyrimido[5,4-d]pyrimidine}, closely related substituted pyrimido-pyrimidines and their preparation have been described in e.g.

U.S. Pat. No. 3,031,450, by Fischer et al. Apr. 24, 1962. Further related substituted pyrimido-pyrimidines and their preparation have been described in e.g. GB 1,051,218, by Thomae, issued Dec. 14, 1966 inter alia, the compound mopidamol {2,6-bisdiethanolamino)-4-piperidinopyrimido[5,4-d]pyrimidine}. Dipyridamole was introduced as a coronary vasodilator in the early 1960s. It is also well known as having platelet aggregation inhibitor properties due to the inhibition of adenosine uptake. Subsequently, dipyridamole was shown to reduce thrombus formation in a study of arterial circulation of the brain in a rabbit model. These investigations led to its use as an antithrombotic agent; it soon became the therapy of choice for such applications as stroke prevention, maintaining the patency of coronary bypass and valve replacement, as well as for treatment prior to coronary angioplasty.

Furthermore, the European Stroke Prevention Study 2 (Diener, H. C. et al., ESPS-2; J. Neurol. Sci., 1996 143(1-2):1-13; Diener, H. C. et al., Neurology, 1998, 51(3):17-19) proved that treatment by dipyridamole alone was as effective as low-dose aspirin in the reduction of stroke risk, and combination therapy with dipyridamole and aspirin was more than twice as effective as aspirin alone.

Dipyridamole appears to inhibit thrombosis through multiple mechanisms. Early studies showed that it inhibits the uptake of adenosine, which was found to be a potent endogenous antithrombotic compound. Dipyridamole was also shown to inhibit cyclic AMP phosphodiesterase, thereby increasing intracellular c-AMP.

Laboratory models reflecting the complex physiology of the blood vessel have shown that the vasculature is not a passive conduit, but interacts profoundly with the blood through an intricate system of checks and balances to protect its integrity after vascular accident. Therefore, the endothelium produces prostacyclin, a potent inhibitor of aggregation. The normal endothelium is not thrombogenic and prevents the attachment of platelets. Various stimulants precipitate the release of endothelium-derived relaxing factor (EDRF), which inhibits platelet adhesion and aggregation. At the same time, intracellular increase in cGMP was shown to be responsible for relaxation of smooth muscle cells following administration of nitro compounds. Thus, the endothelium can inhibit thrombus formation by two separate mechanisms, one mediated by prostacyclin and c-AMP, and the other by EDRF and c-GMP. Dipyridamole appears to enhance both of these antithrombotic mechanisms of the vessel wall in addition to its adenosine-sparing effects. Dipyridamole stimulates prostacyclin production by increasing intracellular levels of cAMP, and it enhances the strongly antithrombotic nitric oxide system by increasing cGMP.

Dipyridamole also has antioxidant properties (Iuliano, L. et al., Free Radic. Biol. Med. 1995, 18:239-247,) that may contribute to its antithrombotic effect. When oxidized, low density lipoproteins become recognized by the scavenger receptor on macrophages, which is assumed to be the necessary step in the development of atherosclerosis (Parthasarathy, S. et al., Ann. Rev. Med. 1992, 43:219-25).

The inhibition of free radical formation by dipyridamole has been found to inhibit fibrinogenesis in experimental liver fibrosis (Wanless, I. R. et al., Hepatology, 1996, 24(4):855-864) and to suppress oxygen radicals and proteinuria in experimental animals with aminonucleoside nephropathy (Nakamura, K. et al., Eur. J. Clin. Invest., 1998, 28:877-883; Nagase, M. et al., Renal Physiol., 1984, 7:218-226). Inhibition of lipid peroxidation also has been observed in human normeoplastic lung tissue (De La Cruz, J. P. et al., Gen. Pharmacol., 1996, 27:855-859).

Mopidamol is known to possess antithrombotic and additionally antimetastatic properties.

### SUMMARY OF THE INVENTION

It has now surprisingly been found that dipyridamole and mopidamol exert a protective effect on the vessel wall thereby strongly influencing the interaction of the vessel wall with the fibrin pathway of the coagulation system resulting in an essential reduction of fibrin accumulation after stimulation of clot formation.

It is known that vascular damages accelerate fibrin accumulation since the prothrombinase complex becomes significantly more potent when settled on negatively charged phospholipids of the cellular membrane. By stabilizing the cellular membrane, less negatively charged phosphatidylserines may become exposed on the outer cell membrane, offering fewer opportunities for the prothrombinase complex to bind to the phospholipids, and thereby preventing the prothrombinase complex from operating at its full conversion rate to turn prothrombin into thrombin which is responsible for the conversion of fibrinogen into fibrin.

Platelet accretion and fibrin accumulation are the basic pathways involved in clot formation. It has been shown that the time course of these two pathways differs essentially (Van Ryn, J. et al., Thromb. Haemost., 1993, 69 (Abstr.):569) proving that both mechanisms are not as stringently coupled as it was anticipated. Whereas the activity of dipyridamole and mopidamol as platelet aggregation inhibitor is well known it is a new finding that these agents additionally are inhibitors of fibrin accumulation mediated by their capacity to stabilize cellular membranes of the vessel wall. This effect is especially important in small vessels or capillary vessels where the ratio of vessel wall surface area to blood volume is high, and provides a new approach for treatment and prevention of fibrin-dependent microcirculation disorders. Therefore, dipyridamole and mopidamol may have therapeutic potential in a variety of diseases involving progressive dysfunction of medium and small-sized vessels.

The known vasodilating activity of dipyridamole was generally considered to be more important in the bigger vessels and in the context of short-term treatment or prevention of acute conditions. In using dipyridamole as a stress test agent, it is known that by short-term high-dose infusion of dipyridamole the vascular autoregulation lags behind thereby showing disproportional perfusion. This is used to differentiate lesser increase in blood flow in poststenotic areas compared with bigger increase in healthy segments of the circulation by nuclear imaging or echocardiography. In case of long-term oral treatment, plasma dipyridamole as well as correlated adenosine levels increase over a period of several hours allowing the autoregulatory systems to compensate whereby under “stress test” conditions dipyridamole plasma levels as well as adenosine levels reach their peaks within four minutes. It has been found that treatment according to the present invention provides a lasting effect on small or capillary vessels and thereby a lasting improvement of microcirculation.

The finding that dipyridamole and mopidamol have significant inhibitory effects on fibrin accumulation via the vessel wall and a stabilizing effect on cell membranes provides a rationale also for combination treatment together with other antithrombotic agents, such as platelet aggregation inhibitors, e.g. acetylsalicylic acid (ASA), clopidogrel or ticlopidine or the pharmaceutically acceptable salts thereof, fibrinogen receptor antagonists (Abciximab, RDGS-peptides, synthetic i.v. or oral fibrinogen antagonists, e.g. fradafiban, lefradafiban or pharmaceutically acceptable salts thereof), heparin and heparinoids or antithrombins, or for combination treatment using additional cardiovascular therapies such as treatment with angiotensin-converting enzyme (ACE) inhibitors, Angiotensin II antagonists, Calcium antagonists or lipid-lowering agents such as the statins.

ASA inhibits aggregation through direct effects on the platelet, in more detail, by irreversibly acetylating platelet cyclooxygenase, thus inhibiting the production of thromboxane, which is strongly thrombotic. In high doses, however, aspirin crosses over into endothelial cells (Pedersen, A. K. and G. A. FitzGerald, New Eng. J. Med., 1984, 311:1206-1211), where it interrupts the production of prostacyclin, a potent natural inhibitor of platelet aggregation and by-product of the “arachidonic cascade” (Moncada, S. and J. R. Vane, New Eng. J. Med., 1979, 300:1142-1147). These observations led to the concept of low-dose antiplatelet therapy with ASA to maximize inhibition of thromboxane while minimizing the loss of prostacyclin (Hanley, S. P., et al., Lancet, 1981, 1:969-971). Combination of dipyridamole or mopidamol according to the invention with the low-dose ASA concept is a preferred embodiment.

In one aspect, the present invention provides a method of treatment of the human or non-human animal body, preferably mammalian body, for treating or preventing fibrin-dependent microcirculation disorders or disease states where such microcirculation disorders are involved, said method comprising administering to said body an effective amount of a pharmaceutical composition comprising a pyrimido-pyrimidine selected from dipyridamole, mopidamol and the pharmaceutically acceptable salts thereof, dipyridamole being preferred, optionally in combination with one or more other antithrombotic agents.

In a different aspect, the present invention provides the use of a pyrimido-pyrimidine selected from dipyridamole, mopidamol and the pharmaceutically acceptable salts thereof, dipyridamole being preferred, optionally in combination with one or more other antithrombotic agents, for the manufacture of a pharmaceutical composition for the treatment of the human or non-human animal body, preferably mammalian body, for treating or preventing fibrin-dependent microcirculation disorders or disease states where such microcirculation disorders are involved.

### SUMMARY OF THE FIGURES

FIG. 1: Simultaneous detection of ⁹⁹Tc labeled platelets and ¹²³I-labeled fibrinogen at one minute intervals after angioplasty of the common carotid artery of rabbits. Control (no treatment) group (N=6) showed after injury a rapid increase of platelets and a gradual build up of fibrinogen. Treatment with heparin (100 U/kg bolus followed by 25 U/kg/h infusion) showed reduction of platelet as well as fibrinogen accretion. No injury measurements showing constant radioactivity.

FIG. 2: Deposition of radioactive labeled platelets (⁹⁹Tc) and fibrinogen (¹²³I) at angioplasty site after treatment with dipyridamole (0.25 mg/kg followed by 0.45 mg/kg/hr). Platelet deposition was reduced, but fibrinogen accretion was almost entirely blocked during the first four hours after angioplasty.

### DETAILED DESCRIPTION OF THE INVENTION

The invention provides a new approach for the treatment and prevention of fibrin-dependent microcirculation disorders associated with progressive dysfunction of medium and small sized vessels comprising administering to a mammal in need of such treatment an effective amount of a pharmaceutical composition containing a pyrimido-pyrimidine selected from dipyridamole, mopidamol and the pharmaceutically acceptable salts thereof. Preferably, the mammal is a human.

Fibrin-dependent microcirculation disorders are meant to be such disorders where fibrin deposition is involved in pathogenesis or progression of dysfunction of medium or small-sized vessels leading to a variety of clinical pictures. Metabolic diseases such as diabetes mellitus are known to cause said microcirculation disorders. In addition, inflammatory reactions may cause microcirculation disorders due to local fibrinogen release from the tissue site of inflammation. Furthermore, it is assumed that microcirculation disorders also can also be caused by autoimmune reactions.

The indication “fibrin-dependent microcirculation disorders” should be understood in a non-limiting manner to comprise:

microcirculation disorders caused by metabolic diseases where vascular damages are involved,

such as diabetic angiopathy, especially diabetic microangiopathy, e.g. diabetic gangrene, diabetic retinopathy, diabetic neuropathy or ulcus cruris;

microcirculation disorders caused by inflammatory reactions,

such as morbus crohn,

microcirculation disorders caused by autoimmune diseases,

such as autoimmune chronic-active hepatitis (idiopathic hepatitis), primary-biliary cirrhosisor (autoimmune associated) multiple sclerosis;

peripheral microcirculation disorders,

such as Raynaud's disease, tinnitus or

sudden loss of hearing,

microcirculation disorders associated with increased cell fragmentation,

such as tumor diseases or thrombotic-thrombocytopenic purpura (TTP),

or, as further indications,

nephrosclerosis,

prerenal hypertension,

haemolytic-uremic syndrome (HUS),

arterial hypertension,

vascular dementia,

Alzheimer's disease,

Sudeck's disease,

central-veneous thrombosis of the eye,

ischemic optic neuropathy,

homocystine-induced vasculopathy,

ischemic or coronary heart diseases,

myocardial infarction, reinfarction; and

atherosclerosis.

The indication “fibrin-dependent microcirculation disorders” also includes corresponding disorders of the myocardium. Thus the present invention provides a method for improving the blood supply of the myocardium in a person in need of such treatment, for example, in a person suffering from ischemic or coronary heart disease, as well as a method for prevention of myocardial infarction or reinfarction.

Furthermore, the present invention also provides a method of treatment or prevention of atherosclerosis since administration of dipyridamole or mopidamol supports or helps to improve or restore the microcirculation supplying the vessels.

As already mentioned hereinbefore, dipyridamole, mopidamol or a pharmaceutically acceptable salt thereof can be used alone in a monopreparation or in combination with other antithrombotic agents for the treatment of fibrin-dependent microcirculation disorders.

It is of advantage to maintain a plasma level of dipyridamole or mopidamol of about 0.2 to 5 μmol/L, preferably of about 0.4 to 5 μmol/L, especially of about 0.5 to 2 μmol/L or particularly of about 0.8 to 1.5 μmol/L. This can be achieved using any of the oral dipyridamole sustained release, immediate release, or parenteral formulations on the market, the sustained release formulations being preferred, for instance those available under the trademark Persantin®, or, for the combination therapy with low-dose ASA, using those formulations available under the trademark Asasantin® or Aggrenox®. Dipyridamole sustained release formulations are also disclosed in EP-A-0032562, immediate release formulations are disclosed in EP-A-0068191 and combinations of ASA with dipyridamole are disclosed in EP-A-0257344 which are incorporated by reference. In the case of mopidamol, oral sustained release, immediate release or a parenteral formulations can also be used, e.g. those disclosed in GB 1,051,218 or EP-A-0,108,898 which are incorporated by reference, sustained release formulations being preferred.

Dipyridamole or mopidamol can be administered orally in a daily dosage of 25 to 450 mg, preferably 50 to 240 mg, more preferrably 75 to 200 mg. For long-term treatment, it is of advantage to administer repeated doses such as a dose of 25 mg dipyridamole sustained release or any other immediate release formulation three or four times a day. For parenteral administration, dipyridamole could be given in a dosage of 0.5 to 5 mg/kg body weight, preferably 1 to 3.5 mg/kg body weight, during 24 hours as slow i.v. infusion (not faster than 0.2 mg/min).

Dipyridamole or mopidamol in combination with low-dose ASA may be administered orally in a daily dosage of 10 to 30 mg of ASA together with 50 to 300 mg of dipyridamole or mopidamol, preferably 80 to 240 mg of dipyridamole or mopidamol, for instance in a weight ratio between 1 to 5 and 1 to 12, more preferably a weight ratio of 1 to 8, for instance 25 mg of ASA together with 200 mg of dipyridamole or mopidamol.

Other antithrombotic compounds can be given at 0.1 to 10 times, preferably at 0.3 to 5.0 times, more preferably at 0.3 to 2.0 times the clinically described dose (e.g. Rote Liste® 1999; fradafiban, lefradafiban: EP-A-0483667, together with a daily dosage of 25 to 450 mg, preferably 50 to 240 mg, more preferably 75 to 200 mg of dipyridamole or mopidamol.

For combination treatment using dipyridamole or mopidamol together with ACE inhibitors, any ACE inhibitor known in the art is suitable, e.g. benazepril, captopril, ceronapril, enalapril, fosinopril, imidapril, lisinopril, moexipril, quinapril, ramipril, trandolapril or perindopril, using the dosages known in the art, for instance as described in Rote Liste® 1999, Editio Cantor Verlag Aulendorf.

For combination treatment using dipyridamole or mopidamol together with Angiotensin II antagonists any Angiotensin II antagonist known in the art is suitable, e.g. the sartans such as candesartan, eprosartan, irbesartan, losartan, telmisartan, valsartan, olmesartan or tasosartan, using the dosages known in the art, for instance as described in Rote Liste® 1999, Editio Cantor Verlag Aulendorf.

For combination treatment using dipyridamole or mopidamol together with calcium antagonists, any calcium antagonist known in the art is suitable, e.g. nifedipine, nitrendipine, nisoldipine, nilvadipine, isradipine, felodipine or lacidipine, using the dosages known in the art, for instance as described in Rote Liste® 1999, Editio Cantor Verlag Aulendorf.

For combination treatment using dipyridamole or mopidamol together with statins, any statin known in the art is suitable, e.g. lovastatin, simvastatin, pravastatin, fluvastatin, atorvastatin or cerivastatin, using the dosages known in the art, for instance as described in Rote Liste® 1999, Editio Cantor Verlag Aulendorf.

It should be noted that such microcirculation disorders associated with increased cell fragmentation, as mentioned hereinbefore, especially accelerate fibrin accumulation due to the potentiated free cellular membrane areas activating the prothrombinase complex. These microcirculation disorders, for example, tumor diseases or thrombotic-thrombocytopenic purpura, are preferably treated using high doses of dipyridamole or mopidamol. This means that a plasma level of dipyridamole or mopidamol of about 0.2 to 50 μmol/L, preferably of about 0.5 to 20 μmol/L, more preferably of about 0.5 to 10 μmol/L should be maintained, preferably by slow i.v. infusion. For oral treatment of these indications, dipyridamole or mopidamol should be administered in a daily dosage of about 150 to 1000 mg, preferably 200 to 800 mg, more preferably 200 to 600 mg.

Microcirculation disorders associated with increased cell fragmentation can also be treated by a combination of dipyridamole or mopidamol with low-dose ASA using the high doses of dipyridamole or mopidamol mentioned above, together with an oral daily dosage of about 10 to 30 mg of ASA, preferably of about 25 mg of ASA.

### EXAMPLES

In order to study the inhibition of fibrin accumulation by dipyridamole, the following experiment was carried out:

Study Using Radiolabeled Platelets and Fibrinogen In Vivo

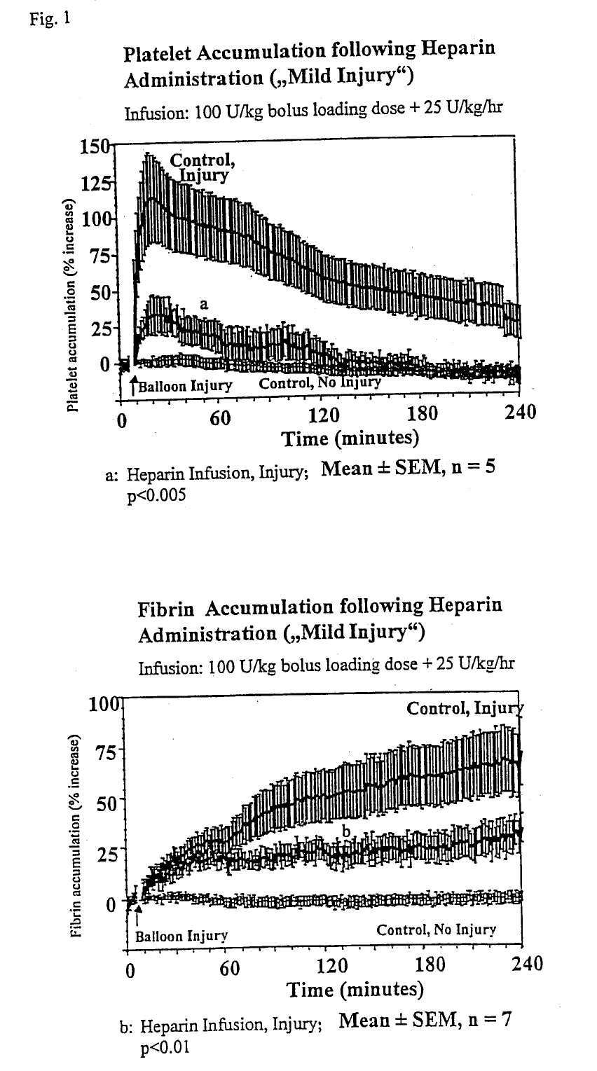

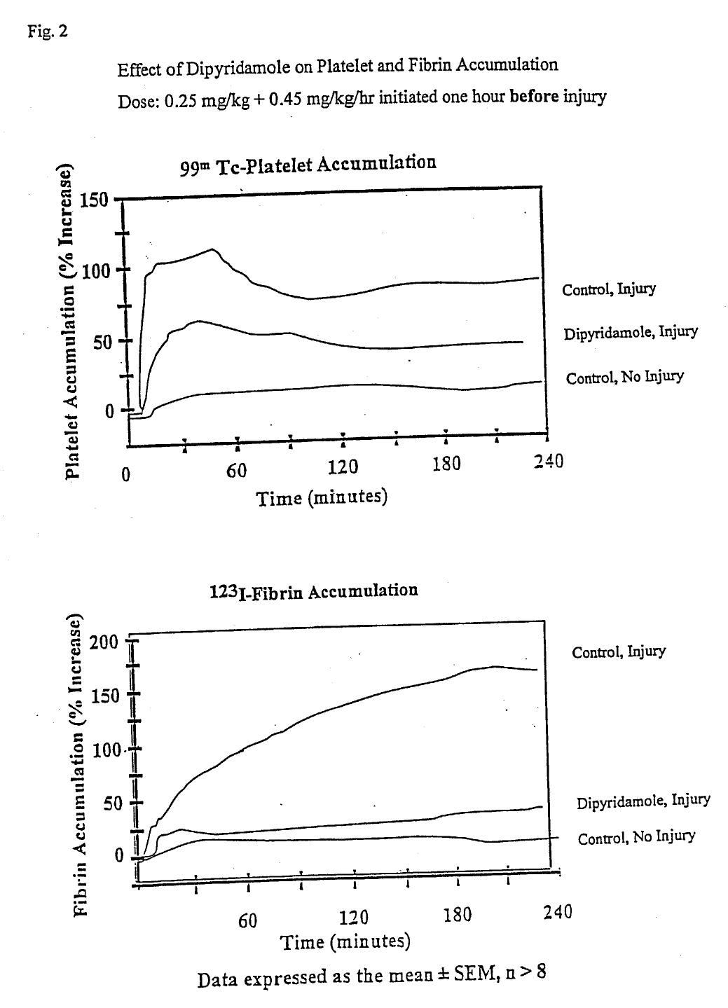

The effects of dipyridamole and heparin were investigated using platelets radiolabeled with ⁹⁹Tc and fibrinogen labeled with ¹²³I. The method is described in Lorenz, M. et al., Nuclear Instruments and Methods in Physics Research A., 1994, 353:448-452. An energy-sensitive solid-state radiation detector was placed to surround each carotid artery in rabbits. After inducing injury through balloon angioplasty, the accretion of platelets and fibrinogen was monitored for four hours. It was found that platelet and fibrinogen accretion had dissimilar time courses. Accretion was not detected in radiolabeled platelets injected 30 minutes after injury; apparently the angioplasty site had been passivated by endogenous platelet adherence. In contrast, fibrinogen accretion did not reach a plateau in control or treated animals even after four hours, indicating that different stimuli or triggers may be regulating fibrinogen at various time points after injury (FIG. 1). Treatment with heparin reduced accumulation of both platelets and fibrinogen. Treatment with dipyridamole also produced a reduction in platelet aggregation which was similar to that seen with heparin. However, the reduction in accretion of fibrinogen was far greater with dipyridamole than with heparin. (FIG. 2).

What is claimed is:

1. A method for treating or preventing in a mammal fibrin-dependent microcirculation disorders or disease states where such microcirculation disorders are involved comprising administering to said mammal an effective amount of a pharmaceutical composition comprising a pyrimido-pyrimidine selected from dipyridamole, mopidamol, and the pharmaceutically acceptable salts thereof alone in a monopreparation.

2. A method for treating in a mammal fibrin-dependent microcirculation disorders or disease states where such microcirculation disorders are involved comprising administering to said mammal an effective amount of a pharmaceutical composition comprising a pyrimido-pyrimidine selected from dipyridamole, mopidamol, and the pharmaceutically acceptable salts thereof in combiation with an agent selected from the group consisting of: acetylsalicylic acid (ASA), clopidogrel, ticlopidine and the pharmaceutically acceptable salts thereof, fibrinogen receptor antagonists, heparin, heparinoids, antithrombins, ACE inhibitors, Angiotensin II antagonists, calcium-antagonists and lipid-lowering agents; wherein ASA is administered orally in a daily dosage of 10 to 30 mg.

3. The method of claim 2 wherein the agent is acetylsalicylic acid (ASA), clopidogrel or ticlopidine or the pharmaceutically acceptable salts thereof.

4. The method of claim 2 wherein the agent is a lipid-lowering agent.

5. The method of claim 4 wherein the lipid-lowering agent is a statin.

6. The method of claim 1 or 2 wherein the pyrimido-pyrimidine is dipyridamole.

7. The method of claim 1 or 2 wherein the mammal is a human.

8. The method of claim 1 or 2 wherein the fibrin-dependent microcirculation disorder is selected from the group consisting of: microcirculation disorders caused by metabolic diseases where vascular damages are involved, microcirculation disorders caused by inflammatory reactions, microcirculation disorders caused by autoimmune diseases, peripheral microcirculation disorders, and microcirculation disorders associated with increased cell fragmentation.

9. The method of claim 8 wherein the fibrin-dependent microcirculation disorder is selected from the group consisting of: diabetic angiopathy, diabetic microangiopathy, diabetic gangrene, diabetic retinopathy, diabetic neuropathy, ulcus cruris, morbus crohn, autoimmune chronic-active hepatitis, idiopathic hepatitis, primary-biliary cirrhosis, multiple sclerosis, Raynaud's disease, tinnitus, sudden loss of hearing, tumor diseases, thrombotic-thrombocytopenic purpura (TTP), nephrosclerosis, prerenal hypertension, haemolytic-uremic syndrome (HUS), arterial hypertension, vascular dementia, Alzheimer's disease, Sudeck's disease, central-veneous thrombosis of the eye, ischemic optic neuropathy, homocystine-induced vasculopathy, ischemic heart diseases, coronary heart diseases, myocardial infarction, myocardial reinfarction, and atherosclerosis.

10. The method of claim 1 or 2 wherein a plasma level of about 0.2 to 5 μmol/L of the pyrimido-pyrimidine is maintained.

11. The method of claim 1 or 2 wherein the pyrimido-pyrimidine is administered using an oral sustained release formulation, an immediate release formulation, or a parenteral formulation.

12. The method of claim 1 or 2 wherein the pyrimido-pyrimidine is administered orally in a daily dosage of 25 to 450 mg or parenterally in a dosage of 0.5 to 5 mg/kg body weight during 24 hours.

13. The method of claim 2 wherein the pharmaceutical composition comprises the pyrimido-pyrimidine in combination with acetylsalicylic acid (ASA) as the other antithrombotic agent, administered orally in a daily dosage of 10 to 30 mg of ASA together with 50 to 300 mg of the pyrimido-pyrimidine.

14. The method of claim 8 wherein a plasma level of dipyridamole or mopidamol of about 0.2 to 50 μmol/L is maintained when treating a microcirculation disorder associated with increased cell fragmentation.

15. The method of claim 14 further comprising administering an oral daily dosage of about 10 to 30 mg of acetylsalicylic acid (ASA).
